(19) United States
(12) Patent Application Publication
(10) Pub. No.: US 2006/0216779 A1
(43) Pub. Date: Sep. 28, 2006

(54) Method for the detection of post-translational modification activities and device system for carrying out said method

(76) Inventor: Larissa Vasilets, Teutschenthal (DE)

Correspondence Address: WILLIAM COLLARD;COLLARD & ROE, P.C., 1077 NORTHERN BOULEVARD, ROSLYN, NY, 11576, US

(21) Appl. No.: 10/550,929

(22) PCT Filed: Mar. 30, 2004
(86) PCT No.: PCT/DE04/00674

§ 371 Date: Sep. 28, 2005

(30) Foreign Application Priority Data
Apr. 2, 2003 (DE) 103149651

(51) Int. Cl. C12Q 1/37; G06F 19/00

(52) U.S. Cl. 435/23; 702/19

(57) ABSTRACT

The invention relates to a method for the qualitative detection of post-translational modification activities in a small liquid sample from organism cells or test chemicals. Phosphorylation/dephosphorylation of proteins by kinases and phosphatases is an example of a post-translational modification. The method is characterised in that protein fragments or polypeptides are synthesised as sensors. The above comprise moieties (1) and (2), containing charged amino acid groups and a recognition site with one or more modification group(s) (X). The sensor has a specific electrostatic potential distribution and a dipole moment. A modification of the sensors occurs on addition of enzymes, associated with a shift in the molecular electrostatic potential distribution and a change in the dipole moment. The potential shift is a determinator for post-translational modification activity. Several device systems are disclosed for the practical carrying out of the method. The method provides a rapid, highly sensitive and effective method for the detection of various types of post-translational activity, in particular, suitable for biological multi-detection systems (biochips and high throughput screening) and finds application in particular to the development of pharmaceuticals, medical diagnostics, basic research and environmental protection.

This invention concerns a method for the qualitative detection of posttranslational modification activities, i.e. the detection of enzymatic activities which modify already synthesized proteins through the formation of specific groups, for example the phosphate group, and alter their function.

Serious diseases such as cancer, diabetes, arthritis, cardiovascular diseases, hypertension and strokes are caused by a change in protein activities. This invention provides a swift, highly sensitive and efficient method for the detection of the various types of posttranslational activities. The method and electronic systems in accordance with this invention make a major contribution to the field of biological multiple-detection systems for the high throughput of essential tests.

Prior to more technological details, the following contains a series of introductory facts:

Systems and methods for the quick detection of analytes with biological activities, particularly in small liquid samples, are of great importance both in the fields of medicine and pharmacy and additionally in the area of environmental protection. One of the most comprehensive and important groups of cellular activities which are known to be vital for the development of pharmaceutical agents are the activities which are effective within posttranslational modification. These activities, characteristic for all living cells, result in the altered functional properties of modified proteins. The main mechanisms of the posttranslational modification of proteins or polypeptides include phosphorylation, methylation, prenylation, ubiquitination and proteolysis. Various external conditions (stimuli), including the presence of growth factors or the development of pathological states such as changes in cell cycles and the effect of toxins, can temporarily modify the posttranslational state of several intracellular components. This necessitates the rapid development of specific and effective inhibitors or activators of certain posttranslational activities. It is therefore important to develop corresponding samples and methods which permit a reliable and sensitive detection of these activities in multiple-detection systems (microarrays, biochips).

An example of posttranslational modification is the phosphorylation/dephosphorylation of proteins by kinases and phosphatases. Kinases modify proteins by attaching a phosphate group (phosphorylation) to amino acid residues, predominantly serine, threonine or tyrosine. Contrary to this, proteins remove phosphatases, so that these phosphate groups reverse the phosphorylation effect. Changes in the phosphorylation state of proteins regulate enzymatic activities through localization and molecular interactions between proteins in live cells. The general balance of kinase and phosphatases activities in a cell is the fundament of the protein phosphorylation state at any point of time. Generally speaking, the effect of protein kinases and phosphatases is one of the major regulatory mechanisms of protein functions. The most recent findings and analyses of diseases indicating genetic defects of protein kinases highlight over 400 specific pathological conditions which can be viewed as being associated with the altered activities of the kinases themselves.

As abnormal protein phosphorylation is the cause for the occurrence of serious diseases such as cancer, diabetes, arthritis, cardiovascular diseases, hypertension and strokes, new compounds that are capable of inhibiting the phosphorylation activities of protein kinases are of great interest to the pharmaceutical industry.

The development of new effective pharmaceutical agents requires the testing of large quantities of compounds which have been synthesized with the aid of combinatory chemistry. For these purposes, the pharmaceutical industry needs new technology to facilitate the necessary tests in a high-throughput format.

Additionally, for the improvement of the effects of already developed pharmaceutical agents, it is also necessary to test to what extent these influence the protein kinase and phosphatase activities. Example: cyclosporin is an immune system inhibitor which is indispensable in organ transplants. It is only recently that studies have demonstrated that this agent functions through the inhibition of the protein phosphatase PP2B.

### STATE-OF-THE-ART REPORT ON INVENTION

Biochip technology is a well-known and extremely efficient method which has revolutionised medical diagnostics and the development of pharmaceutical agents. For example, with the aid of gene chips, complete transcription patterns of tumours or other tissues can be recorded in a test. The development and manufacture of gene chips has already reached an advanced stage. Gene chips cannot however be used to investigate the modified protein activities which cause the previously mentioned serious diseases. For this reason, it is necessary to develop efficient protein chip technologies (processes for the rapid evaluation of modified protein activities in the multiple-detection format). Due to the high complexity of protein chip technologies, these technologies are currently only available to a limited extent.

### INTERNATIONAL STATE-OF-THE-ART REPORT

Current methods for the detection of kinase activities are typically based on measurements using the incorporation of radioactive phosphorus ³²P in protein substrates. For the application of these methods, it is necessary to use very large quantities of radioactivity for the cells in order to label the entire intracellular ATP pool and ensure that the target protein is radioactively labelled. For the detection of the relative phosphorylation of the target protein, it is necessary to dissect cells after they have been incubated with test substances and purify the target protein. This method requires a large number of cells, long incubation periods and careful treatment procedures to avoid incorrect phosphorylation/dephosphorylation results. In addition, this type of procedure requires the purification of the target protein. As the final phosphorylation of the target protein can be very low, this method has a poor degree of efficiency. What is particularly serious for environmental and health issues is the necessity of a high usage of radioactive material during the high-throughput tests according to this method.

Alternative methods for detecting kinase activities are based on phosphorylation-specific antibodies such as ELISA and Western blots. The disadvantage of this method is that it is both difficult and cost-intensive to produce the antibodies and distinguish between the phosphorylated and dephosphorylated protein states.

Mass spectrometry (MALDI and ESI) had already been used for the detection of the primary, structures of proteolysed protein fragments as early as 15 years ago. Theoretically, the mass resolution of this method would be sufficient for the detection of phosphorylation with a mass difference of 80 Da. However, based on the instability of the phosphate bond and its rapid cleavage from protein residues during the measuring procedure, MALDI-MS can only be used for detecting phosphorylation under certain circumstances. The mass-spectrometric detection of protein phosphorylation often leads to erroneous negative results. Additionally, mass spectrometers are extremely cost-intensive.

The U.S. Pat. No. 6,410,255 discloses a method enabling the detection of kinase activity. This method involves a sensor representing a fluorescing group which is inserted into a polypeptide together with a kinase-specific section and a protease-sensitive section. The modification of the protein results in the altered accessibility of the protease cleavage point and cleavage of the fluorescing group. This is detected by means of a fluorescence microscope. In addition to kinases, this method requires the presence of proteases which facilitate the production of artefacts through interaction. Both the high costs of fluorescence microscopes and the necessity for fluorescence labelling of the target protein are disadvantageous.

In addition to the disadvantages mentioned so far in the detection of kinase and phosphatase activities, it can be recapitulated that only limited possibilities exist for the detection of these activities in miniaturized sample volumes containing thousands of components.

Here is a brief summary of the patent DE 100 51 252 A1 with the description “bio chip”. This DE patent describes a procedure for the qualitative and/or quantitative detection of an analyte exhibiting polarizing molecules, particularly biomolecules dissolved in molecular form. A sensor with numerous small measuring cells (with a diameter of <1 mm), each representing a capacitor, exhibits a certain capacity as long as a substrate is contained in the measuring cells. Should supplementary material be added to the substrate containing the molecules to be detected, this will alter the capacity of the capacitor. This alteration in capacity is linked to the concentration of the analyte. Problems in the collection of precise measuring data can be observed in the insertion of bioactive substances into measuring cells. Furthermore, a precision system equipped with spring-loaded contacts for evaluation using a corresponding measuring electronic system occasionally produces measuring errors. There is no rational possibility of rapid detection. This solution according to DE 100 51 252 A1 constitutes a biochip for measuring protein bonds; the detection of posttranslational modification activities is not possible.

It is therefore the task of the invention to develop an automated process for the detection of posttranslational activities which is highly sensitive but uncomplicated in its function and which can be utilised for virtually all kinase and phosphatase activities. No pigments, fluorescent or radioactive substances should be employed.

The swift detection of posttranslational activities and thousands of components should permit a significant contribution to be made in the fields of the research and development of pharmaceutical agents, fundamental research into environmental technology and medicinal molecular diagnostics. The procedure is implemented with the aid of the corresponding electronic systems.

According to the invention, this task will be accomplished as follows; the fundamental concept can be found under the patent claims 1. Further information regarding the invention is contained in the patent claims 2 to 9.

The presentation of the invention necessitates supplementary information.

The technical solution permits the swift and effective detection of posttranslational activities in liquid samples containing analytes or enzymes based on changes in the physicochemical properties of each sensor used. The liquid samples mentioned are samples taken from cells or samples containing test substances to which enzymes have been added.

The sensors developed for detecting posttranslational modification activities consist of synthesized protein fragments or peptides containing a “recognition site” for protein, kinases or other enzymes. These types of sensors will be schematically presented in conjunction with the commentary on design examples in the embodiment of the invention. The “recognition site” is located between two groups of amino acid residues moiety 1 and moiety 2 which possess a quantity of amino acid residues of between 0 and n and a series of charged residues. The sensor is designed such that it possesses a three-dimensional structure with a specific distribution of the molecular electrostatic potential and a molecular dipole moment μ. As a consequence of the above-mentioned posttranslational activities, a modification residue is converted within the recognition site. Correspondingly, the electrostatic potential distribution is altered, resulting in a sensor dipole moment μ*. These changes in the sensor dipole moment are detected by means of electrical and optical methods as will be explained in the design examples.

The following Table 1 shows examples of sensors which can be utilised for the detection of protein kinase A (PKA), protein phosphatase 2B (PP2B), tyrosine kinase (TK), tyrosine phosphatase (TP) and protein kinase C (PKC) activities.

TABLE 1
Primary Structures of Sensors
(bold type indicates modification residues;
italic type indicates recognition site)

| | | | | Sensor |
|---|---|---|---|---|
| Moiety 1 | Recognition Site | Moiety 2 | Activity | No. |
| ELDVPIPGRFD | RRVS | VAAD | PKA | S1 |
| ELDVPIPGRFD | RRVpS | VAAD | PP2B | S2 |
| EI | YETDYY | D | TK | S3 |
| EI | pYETDpYpYp | D | TP | S4 |
| EPEAVAEHG | DKKS | KKAKKER | PKC | S5 |

Sensors with the required three-dimensional structure are designed using molecular modelling utilising bioinformatic methods (e.g. search in Swiss-Prot and PDB databases, structural optimisation using MOE and SYBYL, calculation of the molecular electrostatic potential and dipole moment of unmodified and modified polypeptides). For details, see:

Brandt, W., Anders, A. and Vasilets, L. A. (2002) Predicted alterations in tertiary structure of the N terminus of the Na⁺/K⁺-ATPase α subunit caused by acidic replacement or PKC-mediated phosphorylation of Ser-23. Cell. Biochem. Biophys. 37:83-95.

Example: the dipole moment of the unphosphorylated PKC sensor 5 (S5) according to Table 1 is ca. 203 D, whereby the phosphorylation of serine results in a change in orientation and a reduction of the sensor dipole moment to 144 D.

The above line of reasoning can be summarised as follows:

This invention is based on the detection of posttranslational activities in a liquid sample following changes of the sensor's physicochemical properties without marking the target peptide. Examples for the experimental method for the detection of changes in the sensor dipole moment are measurements of the changes in dielectric constant (permittivity), relaxation currents, refractive index and the density or intensity of polarized light.

The invention will now be explained using design examples and also supplementary notes.

### FIGURES

1 schematic representation of a sensor

2 schematic representation of a device for detecting posttranslational activity utilising optical measurements

3 detection of peptide modification through the measurement of the relative dielectric constant ε

4 detection of posttranslational modification activities utilising differential measurements

5 detection of peptide modification through the measurement of the oscillator frequency shift

In FIG. 1, reference numerals show the following:

1 series of amino acid residues (Moiety 1)

2 series of amino acid residues (Moiety 2)

3 compound

4 binding site

5 solid body

X modification residues

In FIG. 2, reference numerals show the following:

6 light source

7 polariser

8 measuring cell

9 light analyser

10 light detector

P polarized light

In FIG. 3 A, reference numerals show the following:

11 measuring cell

FIG. 4A, reference signs show the following:

12 frequency generator

13 measuring capacitor C1

14 measuring capacitor C2

15 differential amplifier

16 alternating-current/direct-current converter (AC/DC converter)

FIG. 1 is the sensor representing a synthesized polypeptide according to this invention, comprising a “recognition site” for protein kinases or other enzymes. FIG. 1A is the schematic representation of this type of sensor. The “recognition site” with one or more modification residues X is located between two groups of amino acid residues (Moiety 1 and Moiety 2) which possess a series of charged residues. Both the moieties 1 and 2 and the recognition site are structured in such a manner that they lead to a three-dimensional structure with a characteristic distribution of the molecular electrostatic potential. Thus the sensor possesses a molecular dipole moment μ. As a consequence of the above-mentioned posttranslational activities, a modification residue X is converted inside the recognition site into X*. This entails a change in the electrostatic potential distribution and sensor dipole moment μ* (FIG. 1B). These changes in physicochemical sensor properties can be detected through electrical and optical methods (see below).

In an alternative version of this invention, the sensor can be mounted on a solid body 5 coated with Ni-NTA resin with the aid of a flexible connection 3 and a binding group (His-tag) (FIG. 1), representing a glass surface, plastic bead or dielectric.

In a further variant of the invention, the sensor can be directly synthesised on the dielectric-coated solid body surface.

In a third variant, the sensor can be dissolved in molecular form in water.

FIG. 2 shows the schematic representation of a method for the detection of posttranslational activities as a consequence of changes in the optical properties of the sample containing the sensor. A detection cell (measuring cell 8) with a liquid sample and a sensor is located between the source of polarized light \overline{P} and the light analyzer 9. Two thin glass plates are coated on the inside with a gold layer and a dielectric. These serve as contact electrodes. Intensity changes of detected light ΔI are proportional to cos²α (α is the angle of rotation of the polarized light \overline{P}):
ΔI˜cos²α (1)

In the absence of an electric voltage, sensor dipole molecules have a random orientation due to thermal movement. Field E causes the dipoles to line up partially as it competes with the thermal movement of molecules striving to reach a random distribution of dipole orientation according to Boltzmann statistics. Under the normal measurement conditions μE<<kT, the mean moment in field direction can be calculated to be:
\overline{μ}_{E}/μ≅μE/3kT (2)
whereby μ. is the dipole moment of individual molecules, \overline{μ}_{E} is the apparent mean dipole moment in field direction, E—field strength, T—absolute temperature and k=1.3807·10²³ JK⁻¹. As the degree of rotation of polarized light is proportional to the optical activity of a sample with a sensor, which in turn is proportional to the number of molecules oriented in field direction, this yields:
ΔI˜cos²α˜N=\overline{μ}_{E}/μ=μE/3kT (3)

A change in the molecule's dipole moment as a consequence of posttranslational activity leads to changes in the light intensity detected.

According to further variant for the detection of posttranslational activities, the dielectric constant (permittivity) of a liquid sample with a sensor is measured. As is evident from FIG. 3A, the sample is located in a detection cell (measuring cell 11) serving as a capacitor. The introduction of a polarizing dielectric between the capacitor plates reduces the electric field intensity in vacuum E₀ to P/ε₀:
E=E₀−P/ε₀ (4)
where E₀=E/ε and P is the induced polarization of the dielectric with dielectric constant ε.

This yields:
ε=ε₀+P/E (5)

As polarisation means the dipole moment per unit volume, P is therefore proportional to the apparent mean dipole moment \overline{μ}_{E}, which is \overline{μ}_{E}=ρ²E/3kT according to equation (2), resulting for ε in:
ε=ε₀+μ²/kT (6)

Should a dipole moment be altered due to a posttranslational modification, the dielectric constant is changed in compliance with the equation (6).

Should sensor molecules (S) be dissolved in water (W) or other solvents, the molar polarization of the latter has to be considered:
P=P_{W}·x_{W}+P_{S}·x_{S} (7)
whereby x_{S} is the mole fraction of the sensor, x_{S}=n_{S}/(n_{W}+n_{S}) and x_{W} the mole fraction of water, x_{W}=n_{W}/(n_{W}+n_{S}).

FIG. 3B shows an example of the changes in the dielectric constant for two synthesized model peptides S1 and S2 (table 1).

The polypeptides (S1) or (S2) dissolved in water have been placed as a dielectric between the electrode plates of the measuring capacitor (measuring cell 11), see FIG. 3A. Polypeptide ELDVPIPGRFDRRVSVAAD (S1) is a specific substrate for protein kinase A which catalyses the phosphorylation of serine. Phosphatase PP2B implements the dephosphorylation of serine of the polypeptide ELDVPIPGRFDRRVpSVAAD (S2). The dielectric constant of the unphosphorylated polypeptide (S1) ε₃ is 76, while that of the phosphorylated polypeptide (S2) is reduced to 70. These procedures therefore permit the detection of the activities of these enzymes.

Regarding the detection of peptide modification in tests through the measurement of the relative dielectric constant ε, the following should be added: four microlitres of samples, each containing the polypeptide S1 (unphosphorylated) or S2 (phosphorylated) (table 1), dissolved in water to a concentration of 1.14 mM, are directly introduced between the electrode plates of a measuring capacitor. The dielectric constant ε is measured at a temperature of t=23° C. In this example, the minimum sample volume should not be smaller than 4 μm. Protein concentrations will be within the millimole range; this can restrict the utilisation of this measurement procedure for biochips.

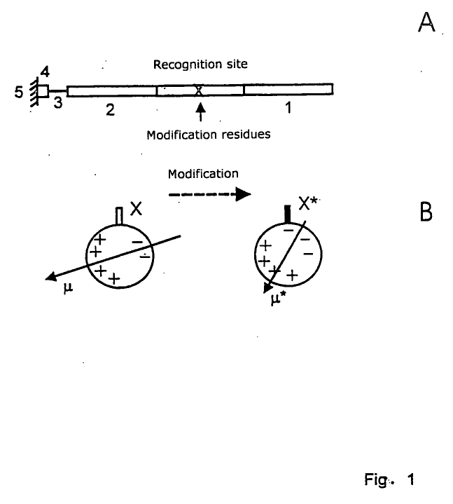

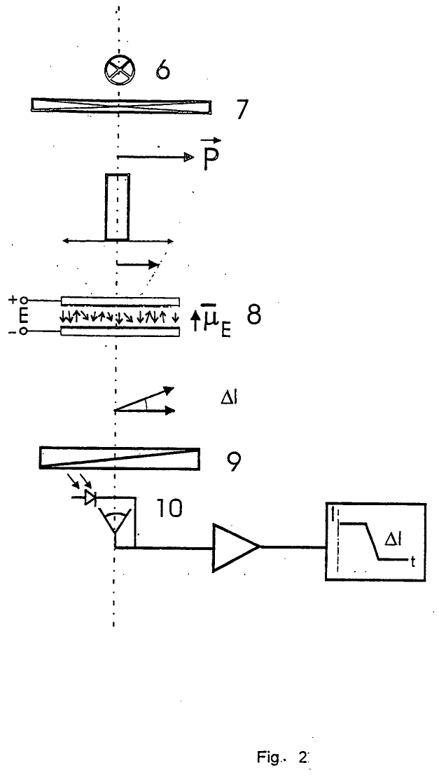

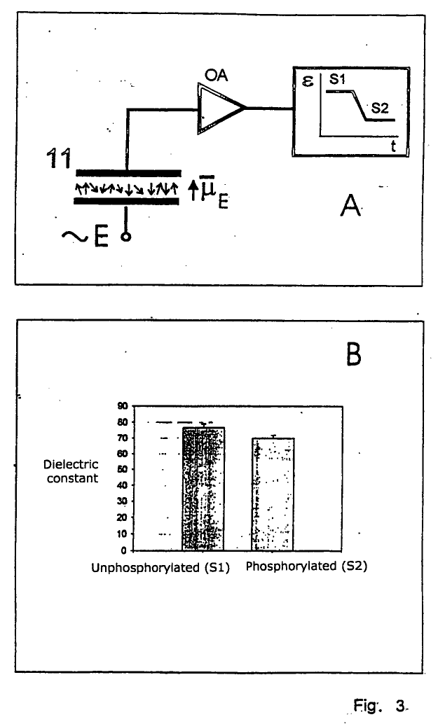

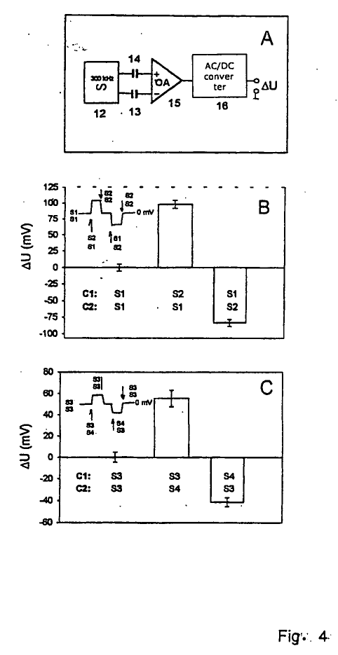

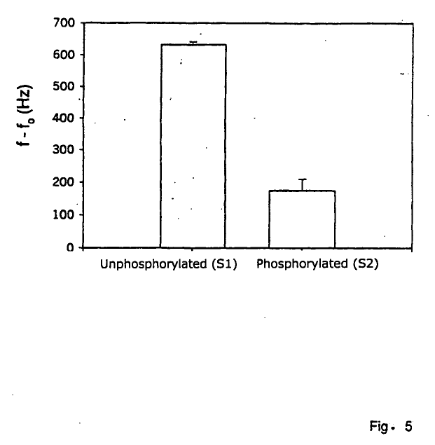

To enable this measurement method to be used for biochips, differential capacitance measurements have been developed which permit the reduction of the sample volume to below 0.5 μL and the protein concentration to 10⁻⁵M. FIGS. 4A and 4B present the results of these differential measurements. Particularly advantageous is the high time resolution of these electrical measurements within the millisecond range and is chiefly dependent on the speed of sample introduction. These methods therefore not only permit the detection of modification activities in small liquid samples, but also the recording of their kinetics.

For details regarding the structure of the device for differential measurements, see FIG. 4A. The measuring device consists of a frequency generator 12, measuring capacitors 13 and 14 which are integrated into a chip plate, an amplifier 15 and an alternating-current/direct-current converter (AC/DC converter) 16. The output signal amplitude which is proportional to the difference of the capacities of the measuring capacitors 13 and 14 is converted into a DC voltage difference AU using the AC/DC converter. This was registered utilising a recording instrument (see FIGS. 4B and 4C) or fed into a digital evaluation system using an analogue-to-digital converter.

FIG. 4B shows the differential measurements with sensors for protein kinase A activity: ELDVPIPGRFDRRVSVAAD (S1) and phosphatase PP2B activity: ELDVPIPGRFDRRVpSVAAD (S2).

FIG. 4C shows the differential measurements with sensors for tyrosine kinase activity EIYETDYY(S3) and for tyrosine phosphatase activity EIpYETDpYpYp (S4).

All sensors are dissolved in water at a concentration of 20 μM and applied either in the measuring capacitor C1 13 or in C2 14 (see labelling). Sample volumes are 0.5 μL. All measurements are carried out at ambient room temperature.

According to a further variant of the method for the detection of posttranslational activities, the polypeptides (S1) or (S2) dissolved in water and used as substrates are located in a measuring cell with an inductivity of L and a capacitance of C which functions as an oscillator (FIG. 5). The frequency ω can be determined by ω=1/LC which can be measured to a high degree of accuracy (10⁻⁵). A distinct reduction in frequency can be observed if the sample contains a phosphorylated polypeptide S2 instead of an unphosphorylated polypeptide S1 (FIG. 5).

For the detection of peptide modification through the measurement of the frequency shift of an oscillator, an experiment was carried out as follows:

A tube containing 150 μL of samples with unphosphorylated peptide S1 or phosphorylated peptide S2 (concentration 1.14M in water) was placed as the core in an induction coil of an oscillator. Frequency f was measured at temperature t=22° C., f₀=5342.9 kHz.

Peptide modification can also be determined through the measurement of the refractive index. According to Maxwell's theory of magnetism, the following relation exists between the relative permittivity and the refractive index n measured at the same frequency:

ε=ε/ε₀=n². This enables alterations in the molecular dipole moment induced by posttranslational activities to be detected through changes in the refractive index:
n=(1+μ²/kTεₒ)^{1/2} (8)

As liquid samples from cells or organisms are extremely complex and contain numerous proteins and minor polarizing molecules, this leads to a high variability in the above-mentioned physicochemical properties of these samples. For the detection of posttranslational activities in complex liquid samples, it is essential that differential measurements be carried out. Dielectric constants, the refractive index or light intensity can for example be detected as the difference between samples with and without a sensor or before and after incubation with a sensor.

### SUMMARY

The above-mentioned method for the detection of posttranslational modification activities enables the detection of protein kinase and phosphatase activities using the electronic systems described above in a solution containing a loose or mounted sensor or protein fragment.

Through the differential capacitance measurement of posttranslational modification activities with variable molecular dipole moments in their modified and unmodified states, the sample volume can be reduced to below 0.5 μL and the sensor peptide concentration to 10⁻⁵M. Consequently, these methods permit the classification of several modification activities and the recording of their kinetics.

The electronic system developed for differential capacitance measurements enables posttranslational modification activities to be detected in an effective, simple, swift and cost-effective manner.

The scope of application for this invention covers the pharmaceutical industry (large- and medium-sized pharmaceutical companies), medical diagnosis and basic research and biotechnological companies with the following targets:
the improvement and optimisation of high-throughput test series for newly synthesized and existent compounds; and
the analysis of their inhibiting effect on protein kinase and phosphatase activities in preclinical studies.

The following special features of the method according to this invention can be highlighted:
the possibility of measuring the kinetics of several posttranslational modification activities and
the creation of prerequisites for measuring highly instable modifications such as the phosphorylation of histamine and arginine amino acid residues.

1. A method for the detection of posttranslational modification activities, wherein the utilisation of protein fragments or polypeptides as sensors created from amino acid residues (moieties 1, 2) which possess a series of charged residues, and a recognition site containing modification residue(s) X and displaying a molecular electrostatic potential distribution, whereby the electrostatic potential distribution is measured as a dipole moment, enzymes are added to the sensor, the dipole moment is measured for a second time and a variation in the electrostatic potential and therefore dipole moment represents the detection of posttranslational modification activities.

2. A method based on claim 1 in which amino acid residues (moieties 1, 2) are present in a quantity from 0 to n and possess a series of charged residues.

3. A method based on claim 1 in which the recognition site in combination with the modification residue/modification residues (X) represents a recognition group only permitting the conversion of the modification residue through the specific proteins kinase or phosphatase.

4. A method based on claim 1 in which the amino acid residues (moieties 1, 2) in combination with the recognition site and modification residue/modification residues (X) display a three-dimensional structure with a distribution of molecular electrostatic potential and molecular dipole moment as prescribed by the manufacturer.

5. A method based on claim 1, in which the synthesized protein fragments are either dissolved in a solution or placed on a solid body (5).

6. A method based on claim 1 in which a change in the molecular electrostatic potential distribution of the protein fragment as a consequence of posttranslational modification activities is measured by the conversion of the electrostatic potential distribution into a different physical measurement unit and is indicated as such, and in which the electrostatic potential distribution in the altered dimensions of the different physical measurement unit is measured and/or recorded.

7. A method based on claim 1 in which posttranslational modification activities are determined on the basis of a differential capacitance measurement through the measurement and/or recording of the changes as ΔU.

8. Electronic system based on claim 1 in which an integral part consists of measuring equipment suitable for the supply of differential measurement results which can calculate the difference in the alterations, to which if necessary a differential amplifier (15) may be connected on the load side, followed by an alternating-current/direct-current converter (16).

9. Electronic system for the implementation of the method based on claim 1 in which changes resulting from modification activities are converted by means of existing components into analogue physical measurement results, and in which these components are connected on their load side with an analogue-to-digital converter for the purpose of digital evaluation.
